(19) United States
(12) Patent Application Publication
(10) Pub. No.: US 2011/0172637 A1
(43) Pub. Date: Jul. 14, 2011

(54) DRUG DELIVERY DEVICE INCLUDING TISSUE SUPPORT STRUCTURE

(75) Inventors: Benjamin J. Moga, Madison, WI (US); Kent Chase, Sun Prairie, WI (US); Garrick D.S. Smith, MAdison, WI (US)

(73) Assignees: Ratio, Inc.; FluGen, Inc.

(21) Appl. No.: 12/684,834

(22) Filed: Jan. 8, 2010

(51) Int. Cl. A61M 5/32

(52) U.S. Cl. 604/506; 604/181; 604/180; 604/173

(57) ABSTRACT

A drug delivery device for delivering a drug to a subject includes a microneedle configured to facilitate delivery of the drug to the subject. The microneedle includes a tip portion and is moveable from an inactive position to an activated position. When the microneedle is moved to the activated position, the tip portion of the microneedle is configured to penetrate the skin of the subject. The drug delivery device includes a tissue support structure that includes a channel and an engagement element. The channel has a first end and a second end and is in axial alignment with the microneedle. At least the tip portion of the microneedle extends past the second end of the channel in the activated position. The engagement element is positioned adjacent to the channel, and the engagement element is configured to engage with the skin of the subject such that the engagement element resists downward deformation of the skin caused by the microneedle as the microneedle moves from the inactive position to the activated position.

### BACKGROUND

The present invention relates generally to the field of drug delivery devices. The present invention relates specifically to active transdermal drug delivery devices including a tissue support structure to facilitate drug delivery and using a microneedle as the point of drug delivery.

An active agent or drug (e.g., pharmaceuticals, vaccines, hormones, nutrients, etc.) may be administered to a patient through various means. For example, a drug may be ingested, inhaled, injected, delivered intravenously, etc. In some applications, a drug may be administered transdermally. In some transdermal applications, such as transdermal nicotine or birth control patches, a drug is absorbed through the skin. Passive transdermal patches often include an absorbent layer or membrane that is placed on the outer layer of the skin. The membrane typically contains a dose of a drug that is allowed to be absorbed through the skin to deliver the substance to the patient. Typically, only drugs that are readily absorbed through the outer layer of the skin may be delivered with such devices.

Other drug delivery devices are configured to provide for increased skin permeability to the delivered drugs. For example, some devices use a structure, such as one or more microneedles, to facilitate transfer of the drug into the skin. Solid microneedles may be coated with a dry drug substance. The puncture of the skin by the solid microneedles increases permeability of the skin allowing for absorption of the drug substance. Hollow microneedles may be used to provide a fluid channel for drug delivery below the outer layer of the skin. Other active transdermal devices utilize other mechanisms (e.g., iontophoresis, sonophoresis, etc.) to increase skin permeability to facilitate drug delivery.

### SUMMARY

One embodiment of the invention relates to a drug delivery device for delivering a drug to a subject. The drug delivery device includes a microneedle configured to facilitate delivery of the drug to the subject. The microneedle includes a tip portion and is moveable from an inactive position to an activated position. When the microneedle is moved to the activated position, the tip portion of the microneedle is configured to penetrate the skin of the subject. The drug delivery device includes a tissue support structure that includes a channel and an engagement element. The channel has a first end and a second end and is in axial alignment with the microneedle. At least the tip portion of the microneedle extends past the second end of the channel in the activated position. The engagement element is positioned adjacent to the channel, and the engagement element is configured to engage with the skin of the subject such that the engagement element resists downward depression and/or deformation of the skin surface caused by the microneedle as the microneedle moves from the inactive position to the activated position.

Another embodiment of the invention relates to a drug delivery device for delivering a liquid drug into the skin of a subject. The drug delivery device includes a drug reservoir for storing a dose of the liquid drug and a microneedle component including a hollow microneedle. The hollow microneedle includes a tip portion and a central channel extending through the tip portion of the hollow microneedle. The microneedle component is moveable from an inactive position to an activated position, and when the microneedle component is moved to the activated position, the tip portion of the hollow microneedle is configured to penetrate the skin of the subject. The drug delivery device includes a drug channel extending from the drug reservoir and coupled to the microneedle component such that the drug reservoir is in fluid communication with the tip portion of the hollow microneedle. The drug delivery device includes an engagement element positioned adjacent to the hollow microneedle in the activated position. The engagement element is configured to adhere to the skin of the subject such that the engagement element exerts reaction forces on the skin perpendicular to and/or in the direction opposite to the movement of the microneedle component from the inactive position to the activated position.

Another embodiment of the invention relates to a method of delivering a drug to the skin of a subject. The method includes providing a drug delivery device. The drug delivery device includes a dose of the drug to be delivered, at least one microneedle, an attachment element and a tissue support structure including a skin engagement element. The method includes attaching the drug delivery device to the skin of the subject via the attachment element and attaching the skin engagement element to the skin of the subject. The method includes moving the microneedle from an inactive position to an activated position in which a tip portion of the microneedle pierces the skin of the subject. The method includes limiting surface deformation in a portion of the skin located beneath the microneedle via the skin engagement element facilitating piercing of the skin by the microneedle. The method includes delivering the dose of drug to the subject via the microneedle.

Another embodiment of the invention relates to a drug delivery device for delivering a drug to a subject. The device includes a microneedle component having a body and a microneedle. The microneedle is configured to facilitate delivery of the drug to the subject. The microneedle includes a tip portion, and the microneedle is moveable from an inactive position to an activated position. When the microneedle is moved to the activated position, the tip portion of the microneedle is configured to penetrate the skin of the subject. The device includes a housing having a bottom wall, and a channel defined in the bottom wall. The channel has a first end and a second end, and the channel is aligned with the microneedle. At least the tip portion of the microneedle extends past the second end of the channel in the activated position, and at least a portion of the body of the microneedle component bears against a surface of the bottom wall in the activated position.

Alternative exemplary embodiments relate to other features and combinations of features as may be generally recited in the claims

### BRIEF DESCRIPTION OF THE FIGURES

This application will become more fully understood from the following detailed description, taken in conjunction with the accompanying figures, wherein like reference numerals refer to like elements in which:

FIG. 1 is a perspective view of a drug delivery device assembly having a cover and a protective membrane according to an exemplary embodiment;

FIG. 2 is a perspective view of a drug delivery device according to an exemplary embodiment after both the cover and protective membrane have been removed;

FIG. 3 is a exploded perspective view of a drug delivery device assembly according to an exemplary embodiment;

FIG. 4 is a exploded perspective view of a drug delivery device showing various components mounted within the device housing according to an exemplary embodiment;

FIG. 5 is a exploded perspective view of a drug delivery device showing various components removed from the device housing according to an exemplary embodiment;

FIG. 6 is a perspective sectional view showing a drug delivery device prior to activation according to an exemplary embodiment;

FIG. 7 is a perspective sectional view showing a drug delivery device following activation according to an exemplary embodiment;

FIG. 8 is a side sectional view showing a drug delivery device following activation according to an exemplary embodiment;

FIG. 9 is a side sectional view showing a drug delivery device following delivery of a drug according to an exemplary embodiment;

FIG. 10 is a exploded view showing a portion of a drug delivery device including a tissue support structure according to an exemplary embodiment;

FIG. 11 is an enlarged sectional view showing a portion of a drug delivery device according to an exemplary embodiment following activation;

FIG. 12 is an enlarged sectional view showing a portion of a drug delivery device adhered to the skin prior to activation according to an exemplary embodiment;

FIG. 13 is an enlarged sectional view showing a portion of a drug delivery device adhered to the skin during activation according to an exemplary embodiment;

FIG. 14 is an enlarged view showing a microneedle during activation according to an exemplary embodiment;

FIG. 15 is an enlarged sectional view showing a portion of a drug delivery device adhered to the skin following activation according to an exemplary embodiment;

FIG. 16 is an enlarged view showing a microneedle following activation according to an exemplary embodiment;

FIG. 17 is an enlarged sectional view showing a portion of a drug delivery device according to another exemplary embodiment following activation;

FIG. 18 is a exploded view showing a portion of a drug delivery device including a tissue support structure according to another exemplary embodiment; and

FIG. 19 is a exploded view showing a portion of a drug delivery device including a tissue support structure according to another exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring generally to the figures, a substance delivery device assembly is shown according to various exemplary embodiments. The delivery device assembly includes various packaging and/or protective elements that provide for protection during storage and transportation. The assembly also includes a substance delivery device that is placed in contact with the skin of a subject (e.g., a human or animal, etc.) prior to delivery of the substance to the subject. After the device is affixed to the skin of the subject, the device is activated in order to deliver the substance to the subject. Following delivery of the substance, the device is removed from the skin.

The delivery device described herein may be utilized to deliver any substance that may be desired. In one embodiment, the substance to be delivered is a drug, and the delivery device is a drug delivery device configured to deliver the drug to a subject. As used herein the term “drug” is intended to include any substance delivered to a subject for any therapeutic, preventative or medicinal purpose (e.g., vaccines, pharmaceuticals, nutrients, nutraceuticals, etc.). In one such embodiment, the drug delivery device is a vaccine delivery device configured to deliver a dose of vaccine to a subject. In one embodiment, the delivery device is configured to deliver a flu vaccine. The embodiments discussed herein relate primarily to a device configured to deliver a substance intradermally. In other embodiments, the device may be configured to deliver a substance transdermally or may be configured to deliver drugs directly to an organ other than the skin.

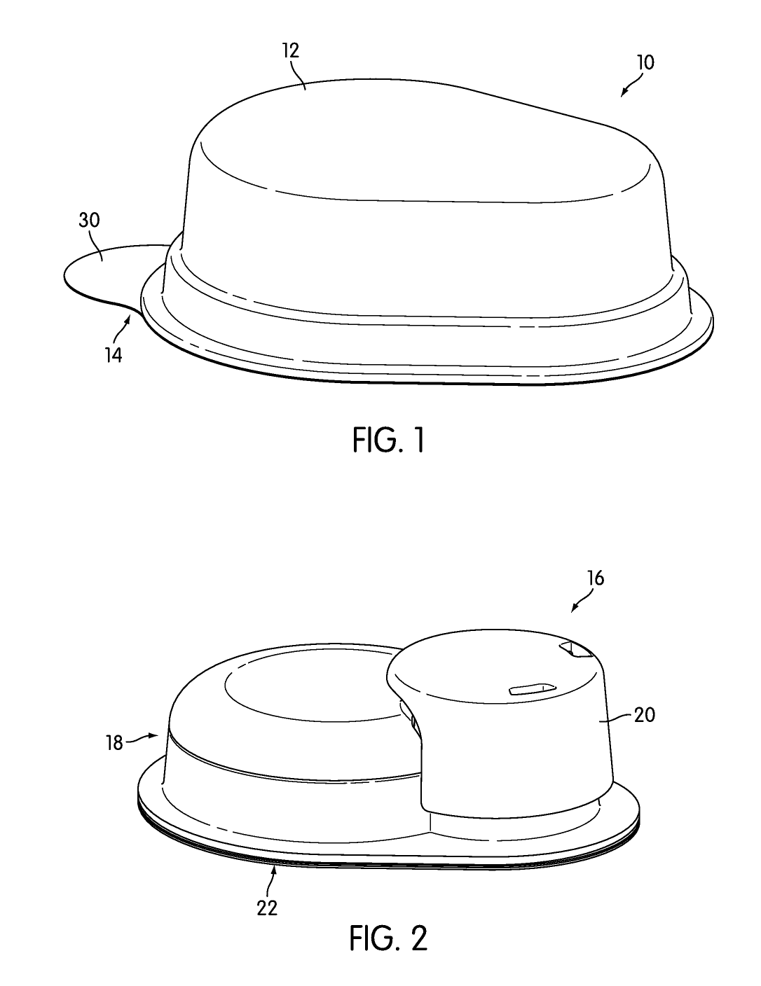

Referring to FIG. 1, drug delivery device assembly 10 is depicted according to an exemplary embodiment. Drug delivery device assembly 10 includes an outer protective cover 12 and a protective membrane or barrier 14 that provides a sterile seal for drug delivery device assembly 10. As shown in FIG. 1, drug delivery device assembly 10 is shown with cover 12 and protective barrier 14 in an assembled configuration. Generally, cover 12 and protective barrier 14 protect various components of drug delivery device 16 during storage and transport prior to use by the end user. In various embodiments, cover 12 may be made of a relatively rigid material (e.g., plastic, metal, cardboard, etc.) suitable to protect other components of drug delivery device assembly 10 during storage or shipment. As shown, cover 12 is made from a non-transparent material. However, in other embodiments cover 12 is a transparent or semi-transparent material.

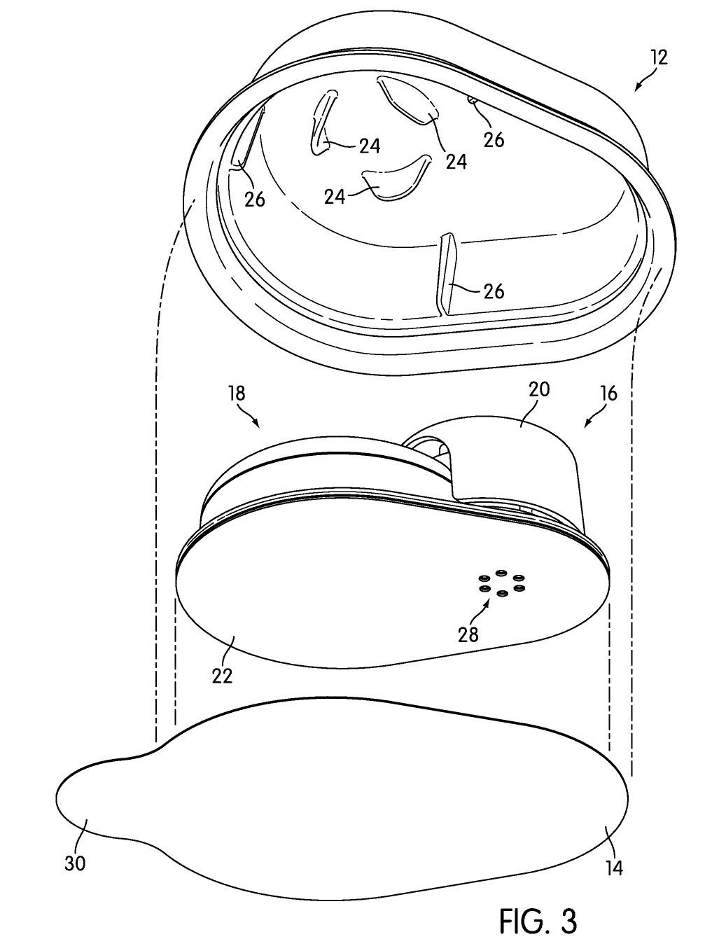

As shown in FIG. 2 and FIG. 3, the drug delivery device assembly includes delivery device 16. Delivery device 16 includes a housing 18, an activation control, shown as, but not limited to, button 20, and an attachment element, shown as, but not limited to, adhesive layer 22. Adhesive layer 22 includes one or more holes 28 (see FIG. 3). Holes 28 provide a passageway for one or more hollow drug delivery microneedles as discussed in more detail below. During storage and transport, cover 12 is mounted to housing 18 of delivery device 16 such that delivery device 16 is received within cover 12. In the embodiment shown, cover 12 includes three projections or tabs 24 extending from the inner surface of the top wall of cover 12 and three projections or tabs 26 extending from the inner surface of the sidewall of cover 12. When cover 12 is mounted to delivery device 16, tabs 24 and 26 contact the outer surface of housing 18 such that delivery device 16 is positioned properly and held within cover 12. Protective barrier 14 is attached to the lower portion of cover 12 covering adhesive layer 22 and holes 28 during storage and shipment. Together, cover 12 and protective barrier 14 act to provide a sterile and hermetically sealed packaging for delivery device 16.

Referring to FIG. 3, to use delivery device 16 to deliver a drug to a subject, protective barrier 14 is removed exposing adhesive layer 22. In the embodiment shown, protective barrier 14 includes a tab 30 that facilitates griping of protective barrier 14 during removal. Once adhesive layer 22 is exposed, delivery device 16 is placed on the skin. Adhesive layer 22 is made from an adhesive material that forms a nonpermanent bond with the skin of sufficient strength to hold delivery device 16 in place on the skin of the subject during use. Cover 12 is released from delivery device 16 exposing housing 18 and button 20 by squeezing the sides of cover 12. With delivery device 16 adhered to the skin of the subject, button 20 is pressed to trigger delivery of the drug to the patient. When delivery of the drug is complete, delivery device 16 may be detached from the skin of the subject by applying sufficient force to overcome the grip generated by adhesive layer 22.

In one embodiment, delivery device 16 is sized to be conveniently wearable by the user during drug delivery. In one embodiment, the length of delivery device 16 along the device's long axis is 53.3 mm, the length of delivery device 16 along the device's short axis (at its widest dimension) is 48 mm, and the height of delivery device 16 at button 20 following activation is 14.7 mm. However, in other embodiments other dimensions are suitable for a wearable drug delivery device. For example, in another embodiment, the length of delivery device 16 along the device's long axis is between 40 mm and 80 mm, the length of delivery device 16 along the device's short axis (at its widest dimension) is between 30 mm and 60 mm, and the height of delivery device 16 at button 20 following activation is between 5 mm and 30 mm. In another embodiment, the length of delivery device 16 along the device's long axis is between 50 mm and 55 mm, the length of delivery device 16 along the device's short axis (at its widest dimension) is between 45 mm and 50 mm, and the height of delivery device 16 at button 20 following activation is between 10 mm and 20 mm.

While in the embodiments shown the attachment element is shown as, but not limited to, adhesive layer 22, other attachment elements may be used. For example, in one embodiment, delivery device 16 may be attached via an elastic strap. In another embodiment, delivery device 16 may not include an attachment element and may be manually held in place during delivery of the drug. Further, while the activation control is shown as button 20, the activation control may be a switch, trigger, or other similar element, or may be more than one button, switch, trigger, etc., that allows the user to trigger delivery of the drug.

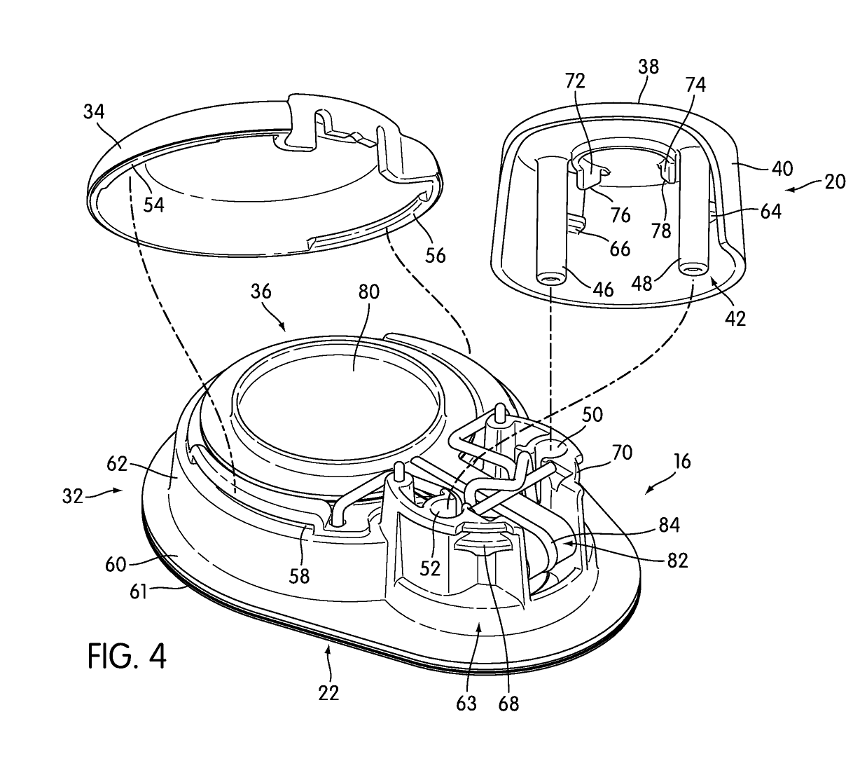

Referring to FIG. 4, housing 18 of delivery device 16 includes a base portion 32 and a reservoir cover 34. Base portion 32 includes a flange 60, a bottom tensile member, shown as bottom wall 61, a first support portion 62 and a second support portion 63. In the embodiment shown, bottom wall 61 is a rigid wall that is positioned below flange 60. As shown in FIG. 4, the outer surface of first support portion 62 is generally cylindrically shaped and extends upward from flange 60. Second support portion 63 is generally cylindrically shaped and extends upward from flange 60 to a height above first support portion 62. As shown in FIG. 4, delivery device 16 includes a substance delivery assembly 36 mounted within base portion 32 of housing 18.

Reservoir cover 34 includes a pair of tabs 54 and 56 that each extend inwardly from a portion of the inner edge of cover 34. Base portion 32 includes a recess 58 and second recess similar to recess 58 on the opposite side of base portion 32. As shown in FIG. 4, both recess 58 and the opposing recess are formed in the upper peripheral edge of the outer surface of first support portion 62. When reservoir cover 34 is mounted to base portion 32, tab 54 is received within recess 58 and tab 56 is received within the similar recess on the other side of base portion 32 to hold cover 34 to base portion 32.

As shown in FIG. 4, button 20 includes a top wall 38. Button 20 also includes a sidewall or skirt 40 that extends from a portion of the peripheral edge of top wall 38 such that skirt 40 defines an open segment 42. Button 20 is shaped to receive the generally cylindrical shaped second support portion 63 of base portion 32. Button 20 includes a first mounting post 46 and a second mounting post 48 both extending in a generally perpendicular direction from the lower surface of top wall 38. Second support portion 63 includes a first channel 50 and a second channel 52. Mounting posts 46 and 48 are slidably received within channels 50 and 52, respectively, when button 20 is mounted to second support portion 63. Mounting posts 46 and 48 and channels 50 and 52 act as a vertical movement guide for button 20 to help ensure that button 20 moves in a generally downward vertical direction in response to a downward force applied to top wall 38 during activation of delivery device 16. Precise downward movement of button 20 ensures button 20 interacts as intended with the necessary components of substance delivery assembly 36 during activation.

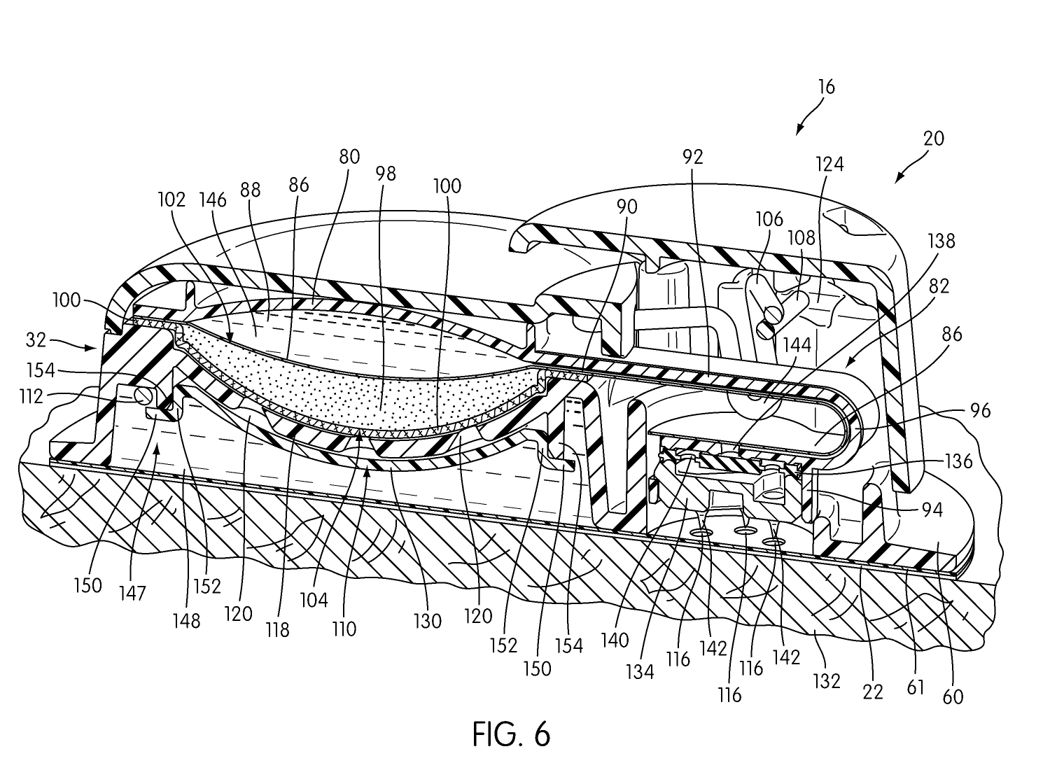

Button 20 also includes a first support ledge 64 and a second support ledge 66 both extending generally perpendicular to the inner surface of sidewall 40. The outer surface of second support portion 63 includes a first button support surface 68 and second button support surface 70. When button 20 is mounted to second support portion 63, first support ledge 64 engages and is supported by first button support surface 68 and second support ledge 66 engages and is supported by second button support surface 70. The engagement between ledge 64 and surface 68 and between ledge 66 and surface 70 supports button 20 in the pre-activation position (shown for example in FIG. 6). Button 20 also includes a first latch engagement element 72 and a second latch engagement element 74 both extending in a generally perpendicular direction from the lower surface of top wall 38. First latch engagement element 72 includes an angled engagement surface 76 and second latch engagement element 74 includes an angled engagement surface 78.

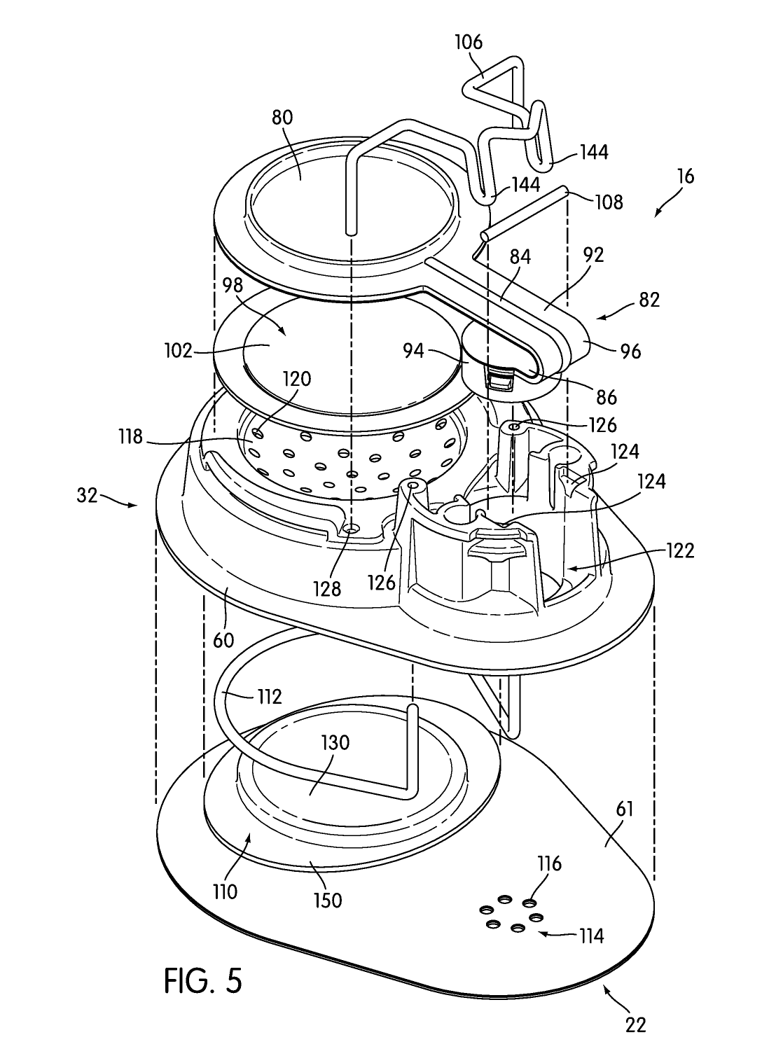

Referring to FIG. 4 and FIG. 5, substance delivery assembly 36 includes a drug reservoir base 80 and drug channel arm 82. The lower surface of drug channel arm 82 includes a depression or groove 84 that extends from reservoir base 80 along the length of drug channel arm 82. As shown in FIG. 4 and FIG. 5, groove 84 appears as a rib protruding from the upper surface of drug channel arm 82. Substance delivery assembly 36 further includes a flexible barrier film 86 adhered to the inner surfaces of both drug reservoir base 80 and drug channel arm 82. Barrier film 86 is adhered to form a fluid tight seal or a hermetic seal with drug reservoir base 80 and channel arm 82. In this arrangement (shown best in FIGS. 6-9), the inner surface of drug reservoir base 80 and the inner surface of barrier film 86 form a drug reservoir 88, and the inner surface of groove 84 and the inner surface of barrier film 86 form a fluid channel, shown as, but not limited to, drug channel 90. In this embodiment, drug channel arm 82 acts as a conduit to allow fluid to flow from drug reservoir 88. As shown, drug channel arm 82 includes a first portion 92 extending from drug reservoir base 80, a microneedle attachment portion, shown as, but not limited to, cup portion 94, and a generally U-shaped portion 96 joining the first portion 92 to the cup portion 94. In the embodiment shown, drug reservoir base 80 and drug channel arm 82 are made from an integral piece of polypropylene. However, in other embodiments, drug reservoir base 80 and drug channel arm 82 may be separate pieces joined together and may be made from other plastics or other materials.

Substance delivery assembly 36 includes a reservoir actuator or force generating element, shown as, but not limited to, hydrogel 98, and a fluid distribution element, shown as, but not limited to, wick 100 in FIG. 6. Because FIG. 5 depicts delivery device 16 in the pre-activated position, hydrogel 98 is formed as a hydrogel disc and includes a concave upper surface 102 and a convex lower surface 104. As shown, wick 100 is positioned below hydrogel 98 and is shaped to generally conform to the convex shape of lower surface 104.

Substance delivery assembly 36 includes a microneedle activation element or microneedle actuator, shown as, but not limited to, torsion rod 106, and a latch element, shown as, but not limited to, latch bar 108. As explained in greater detail below, torsion rod 106 stores energy, which upon activation of delivery device 16, is transferred to one or more microneedles causing the microneedles to penetrate the skin. Substance delivery assembly 36 also includes a fluid reservoir plug 110 and plug disengagement bar 112. Bottom wall 61 is shown removed from base portion 32, and adhesive layer 22 is shown coupled to the lower surface of bottom wall 61. Bottom wall 61 includes one or more holes 114 that are sized and positioned to align with holes 28 in adhesive layer 22. In this manner, holes 114 in bottom wall 61 and holes 28 in adhesive layer 22 form channels, shown as needle channels 116.

As shown in FIG. 5, first support portion 62 includes a support wall 118 that includes a plurality of fluid channels 120. When assembled, wick 100 and hydrogel 98 are positioned on support wall 118 below drug reservoir 88. As shown, support wall 118 includes an upper concave surface that generally conforms to the convex lower surfaces of wick 100 and hydrogel 98. Fluid reservoir plug 110 includes a concave central portion 130 that is shaped to generally conform to the convex lower surface of support wall 118. First support portion 62 also includes a pair of channels 128 that receive the downwardly extending segments of torsion rod 106 such that the downwardly extending segments of torsion rod 106 bear against the upper surface of bottom wall 61 when delivery device 16 is assembled. Second support portion 63 includes a central cavity 122 that receives cup portion 94, U-shaped portion 96 and a portion of first portion 92 of drug channel arm 82. Second support portion 63 also includes a pair of horizontal support surfaces 124 that support latch bar 108 and a pair of channels 126 that slidably receive the vertically oriented portions of plug disengagement bar 112.

Referring to FIG. 6, a perspective, sectional view of delivery device 16 is shown attached or adhered to skin 132 of a subject prior to activation of the device. As shown, adhesive layer 22 provides for gross attachment of the device to skin 132 of the subject. Delivery device 16 includes a microneedle component, shown as, but not limited to, microneedle array 134, having a plurality of microneedles, shown as, but not limited to, hollow microneedles 142, extending from the lower surface of microneedle array 134. In the embodiment shown, microneedle array 134 includes an internal channel 141 allowing fluid communication from the upper surface of microneedle array 134 to the tips of hollow microneedles 142. Delivery device 16 also includes a valve component, shown as, but not limited to, check valve 136. Both microneedle array 134 and check valve 136 are mounted within cup portion 94. Drug channel 90 terminates in an aperture or hole 138 positioned above check valve 136. In the pre-activation or inactive position shown in FIG. 6, check valve blocks hole 138 at the end of drug channel 90 preventing a substance, shown as, but not limited to, drug 146, within drug reservoir 88 from flowing into microneedle array 134. While the embodiments discussed herein relate to a drug delivery device that utilizes hollow microneedles, in other various embodiments, other microneedles, such as solid microneedles, may be utilized.

As shown in FIG. 6, in the pre-activation position, latch bar 108 is supported by horizontal support surfaces 124. Latch bar 108 in turn supports torsion rod 106 and holds torsion rod 106 in the torqued, energy storage position shown in FIG. 6. Torsion rod 106 includes a U-shaped contact portion 144 that bears against a portion of the upper surface of barrier film 86 located above cup portion 94. In another embodiment, U-shaped contact portion 144 is spaced above barrier film 86 (i.e., not in contact with barrier film 86) in the pre-activated position.

Delivery device 16 includes an activation fluid reservoir, shown as, but not limited to, fluid reservoir 147, that contains an activation fluid, shown as, but not limited to, water 148. In the embodiment shown, fluid reservoir 147 is positioned generally below hydrogel 98. In the pre-activation position of FIG. 6, fluid reservoir plug 110 acts as a plug to prevent water 148 from flowing from fluid reservoir 147 to hydrogel 98. In the embodiment show, reservoir plug 110 includes a generally horizontally positioned flange 150 that extends around the periphery of plug 110. Reservoir plug 110 also includes a sealing segment 152 that extends generally perpendicular to and vertically away from flange 150. Sealing segment 152 of plug 110 extends between and joins flange 150 with the concave central portion 130 of plug 110. The inner surface of base portion 32 includes a downwardly extending annular sealing segment 154. The outer surfaces of sealing segment 152 and/or a portion of flange 150 abut or engage the inner surface of annular sealing segment 154 to form a fluid-tight seal preventing water from flowing from fluid reservoir 147 to hydrogel 98 prior to device activation.

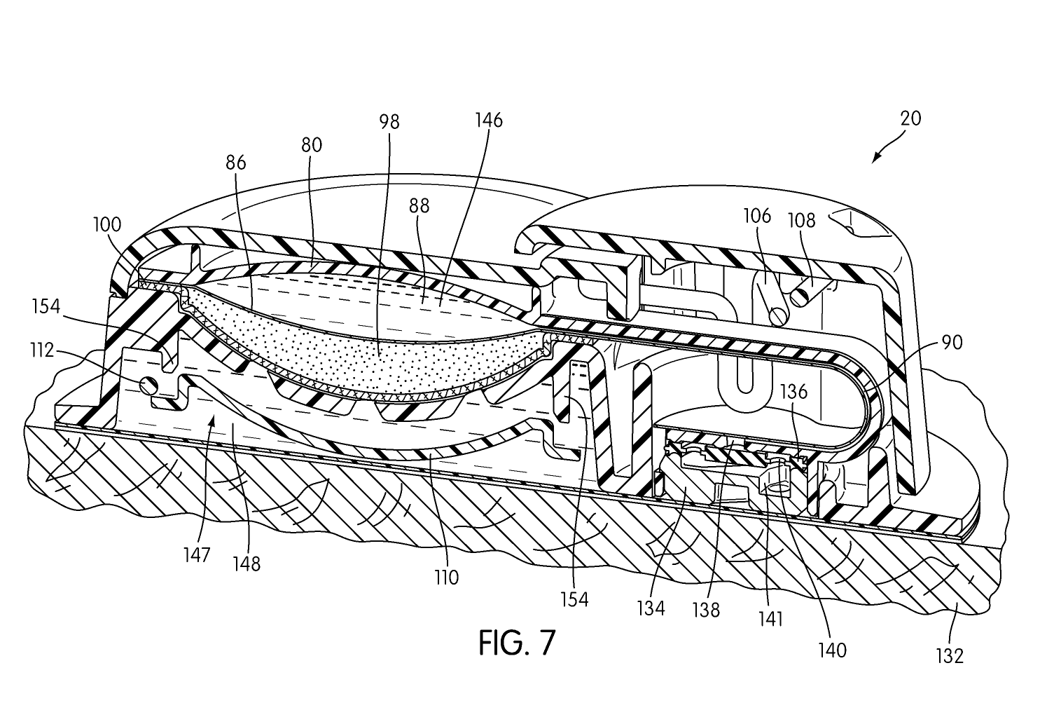

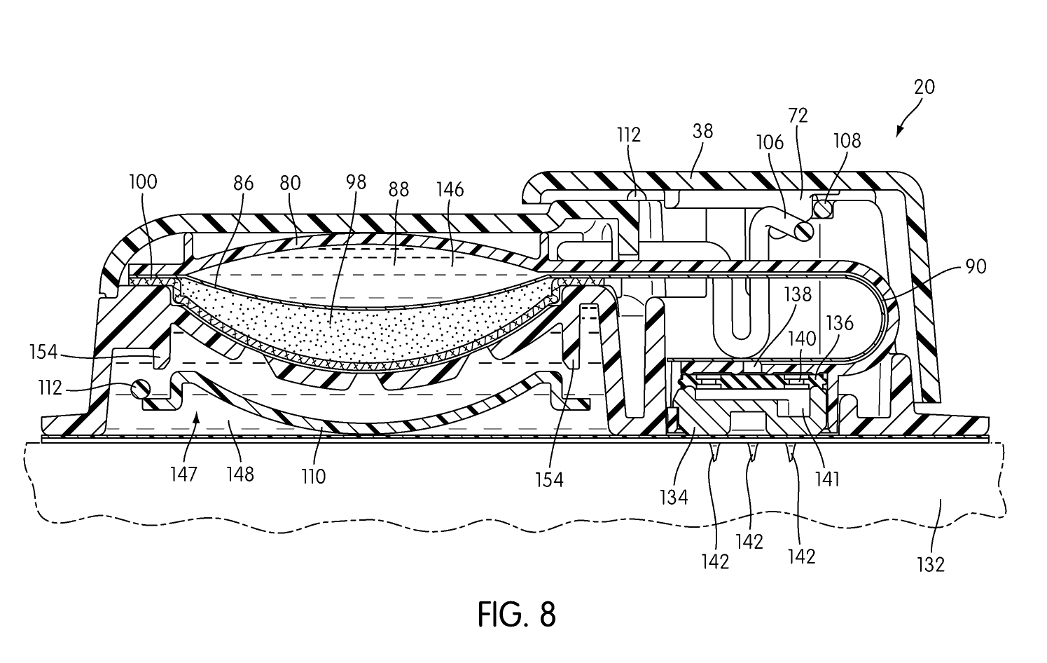

Referring to FIG. 7 and FIG. 8, delivery device 16 is shown immediately following activation. In FIG. 8, skin 132 is drawn in broken lines to show hollow microneedles 142 after insertion into the skin of the subject. To activate delivery device 16, button 20 is pressed in a downward direction (toward the skin). Movement of button 20 from the pre-activation position of FIG. 6 to the activated position causes activation of both microneedle array 134 and of hydrogel 98. Depressing button 20 causes first latch engagement element 72 and second latch engagement element 74 to engage latch bar 108 and to force latch bar 108 to move from beneath torsion rod 106 allowing torsion rod 106 to rotate from the torqued position of FIG. 6 to the seated position of FIG. 7. The rotation of torsion rod drives microneedle array 134 downward and causes hollow microneedles 142 to pierce skin 132. In addition, depressing button 20 causes the lower surface of button top wall 38 to engage plug disengagement bar 112 forcing plug disengagement bar 112 to move downward. As plug disengagement bar 112 is moved downward, fluid reservoir plug 110 is moved downward breaking the seal between annular sealing segment 154 of base portion 32 and sealing segment 152 of reservoir plug 110.

With the seal broken, water 148 within reservoir 147 is put into fluid communication with hydrogel 98. As water 148 is absorbed by hydrogel 98, hydrogel 98 expands pushing barrier film 86 upward toward drug reservoir base 80. As barrier film 86 is pushed upward by the expansion of hydrogel 98, pressure within drug reservoir 88 and drug channel 90 increases. When the fluid pressure within drug reservoir 88 and drug channel 90 reaches a threshold, check valve 136 is forced open allowing drug 146 within drug reservoir 88 to flow through aperture 138 at the end of drug channel 90. As shown, check valve 136 includes a plurality of holes 140, and microneedle array 134 includes a plurality of hollow microneedles 142. Drug channel 90, hole 138, plurality of holes 140 of check valve 136, internal channel 141 of microneedle array 134 and hollow microneedles 142 define a fluid channel between drug reservoir 88 and the subject when check valve 136 is opened. Thus, drug 146 is delivered from reservoir 88 through drug channel 90 and out of the holes in the tips of hollow microneedles 142 to the skin of the subject by the pressure generated by the expansion of hydrogel 98.

In the embodiment shown, check valve 136 is a segment of flexible material (e.g., medical grade silicon) that flexes away from aperture 138 when the fluid pressure within drug channel 90 reaches a threshold placing drug channel 90 in fluid communication with hollow microneedles 142. In one embodiment, the pressure threshold needed to open check valve 136 is about 0.5-1.0 pounds per squire inch (psi). In various other embodiments, check valve 136 may be a rupture valve, a swing check valve, a ball check valve, or other type of valve the allows fluid to flow in one direction. In the embodiment shown, the microneedle actuator is a torsion rod 106 that stores energy for activation of the microneedle array until the activation control, shown as button 20, is pressed. In other embodiments, other energy storage or force generating components may be used to activate the microneedle component. For example, in various embodiments, the microneedle activation element may be a coiled compression spring or a leaf spring. In other embodiments, the microneedle component may be activated by a piston moved by compressed air or fluid. Further, in yet another embodiment, the microneedle activation element may be an electromechanical element, such as a motor, operative to push the microneedle component into the skin of the patient.

In the embodiment shown, the actuator that provides the pumping action for drug 146 is a hydrogel 98 that expands when allowed to absorb water 148. In other embodiments, hydrogel 98 may be an expandable substance that expands in response to other substances or to changes in condition (e.g., heating, cooling, pH, etc.). Further, the particular type of hydrogel utilized may be selected to control the delivery parameters. In various other embodiments, the actuator may be any other component suitable for generating pressure within a drug reservoir to pump a drug in the skin of a subject. In one exemplary embodiment, the actuator may be a spring or plurality of springs that when released push on barrier film 86 to generate the pumping action. In another embodiment, the actuator may be a manual pump (i.e., a user manually applies a force to generate the pumping action). In yet another embodiment, the actuator may be an electronic pump.

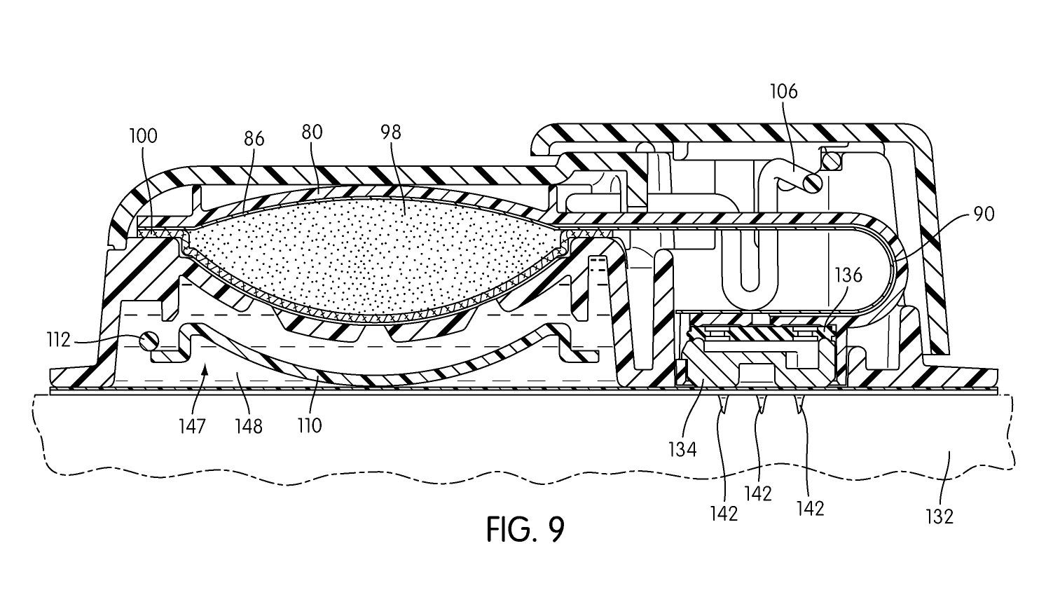

Referring to FIG. 9, delivery device 16 is shown following completion of delivery of drug 146 to the subject. In FIG. 9, skin 132 is drawn in broken lines. As shown in FIG. 9, hydrogel 98 expands until barrier film 86 is pressed against the lower surface of reservoir base 80. When hydrogel 98 has completed expansion, substantially all of drug 146 has been pushed from drug reservoir 88 into drug channel 90 and delivered to skin 132 of the subject. The volume of drug 146 remaining within delivery device 16 (i.e., the dead volume) following complete expansion by hydrogel 98 is minimized by configuring the shape of drug reservoir 88 to enable complete evacuation of the drug reservoir and by minimizing the volume of fluid pathway formed by drug channel 90, hole 138, plurality of holes 140 of check valve 136 and hollow microneedles 142. In the embodiment shown, delivery device 16 is a single-use, disposable device that is detached from skin 132 of the subject and is discarded when drug delivery is complete. However, in other embodiments, delivery device 16 may be reusable and is configured to be refilled with new drug, to have the hydrogel replaced, and/or to have the microneedles replaced.

In one embodiment, delivery device 16 and reservoir 88 are sized to deliver a dose of drug of up to approximately 500 microliters. In other embodiments, delivery device 16 and reservoir 88 are sized to allow delivery of other volumes of drug (e.g., up to 200 microliters, up to 400 microliters, up to 1 milliliter, etc.).

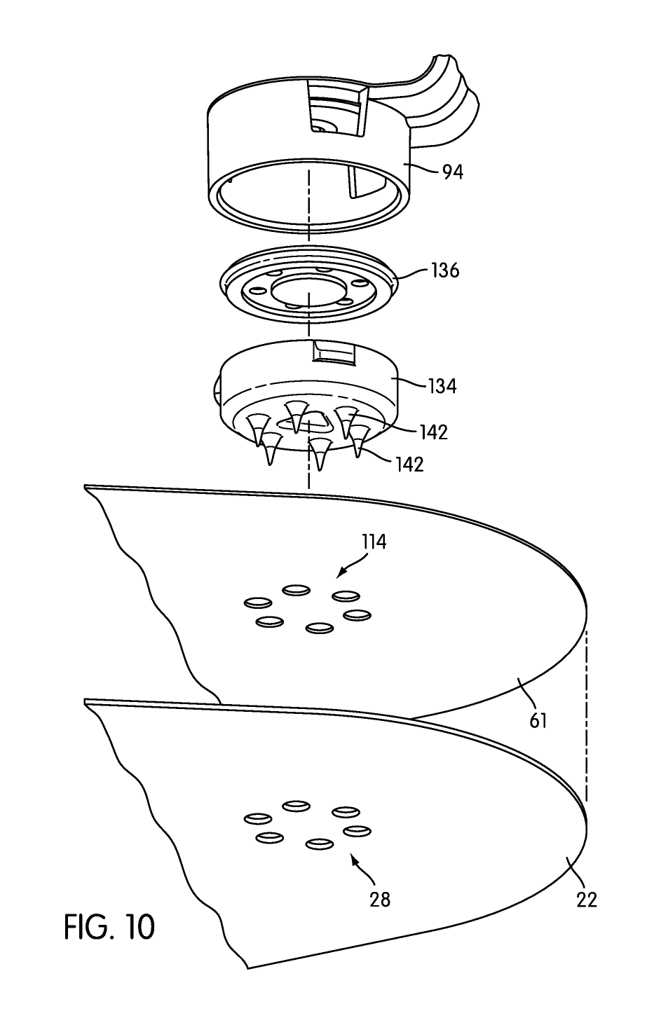

Referring generally to FIGS. 10-19, various embodiments of a substance delivery device including a tissue support structure are shown. Referring specifically to FIG. 10, an exploded view of the microneedle portion of delivery device 16 is shown according to an exemplary embodiment. In the embodiment shown, microneedle array 134 includes six hollow microneedles 142. Check valve 136 is located above microneedle array 134, and, when assembled, both check valve 136 and microneedle array 134 are received within cup portion 94 of channel arm 82. In the embodiment shown, bottom wall 61 includes an array of six holes 114 that correspond to the array of six holes 28 located through adhesive layer 22. When assembled the six microneedles 142 of microneedle array 134 align with holes 114 in bottom wall 61 and with holes 28 in adhesive layer 22.

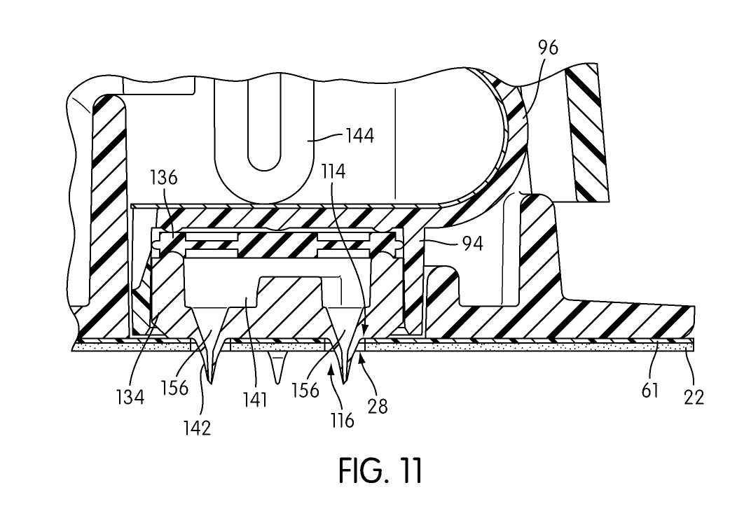

FIG. 11 shows a close-up sectional view of microneedle array 134 and check valve 136 mounted within cup portion 94 after activation of delivery device 16. As shown in FIG. 11, microneedles 142 are cannulated, defining a central channel 156 that places the tip of each microneedle 142 in fluid communication with internal channel 141 of microneedle array 134. As shown in FIG. 11, holes 114 in bottom wall 61 and holes 28 in adhesive layer 22 form a plurality of channels 116. Following activation of microneedle array microneedle array 134 rests against the upper surface of bottom wall 61, and microneedles 142 extend through channels 116. Because bottom wall 61 is constructed of a tensile membrane or rigid material, bottom wall 61 provides a structural backing for adhesive layer 22.

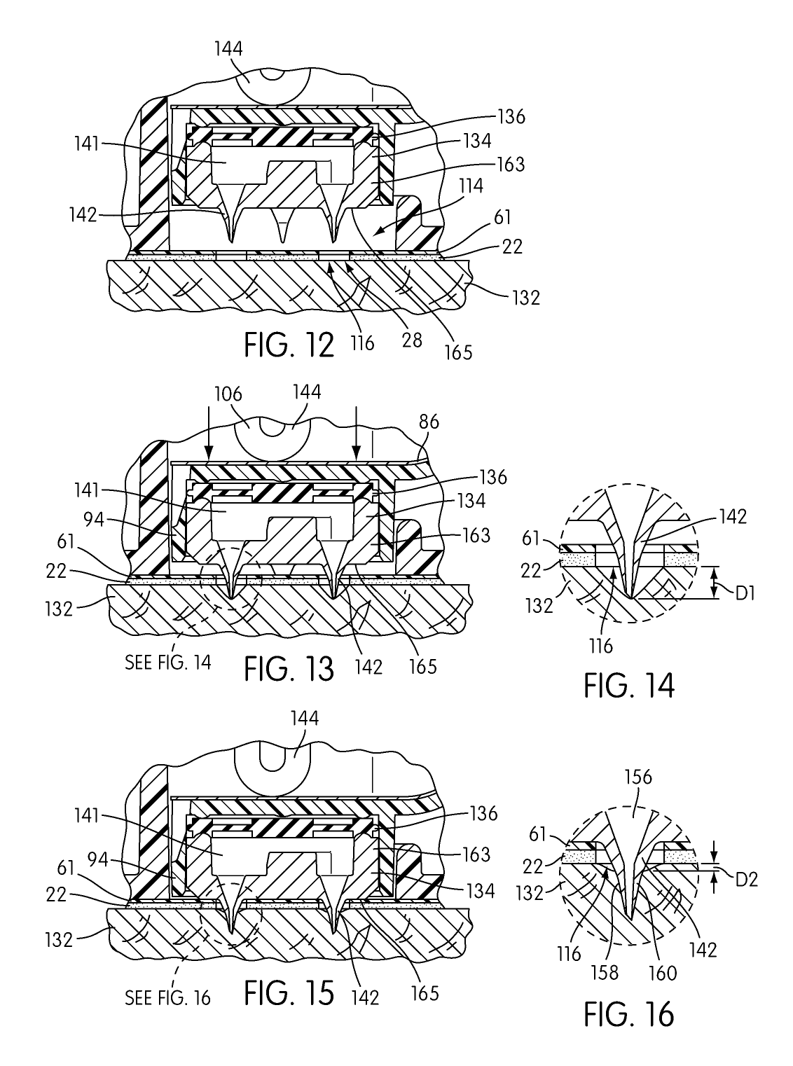

Referring generally to FIGS. 12-16, puncture or penetration of skin 132 by microneedles 142 assisted by a tissue support structure is illustrated according to an exemplary embodiment. When a microneedle is brought into contact with the skin of a subject, the skin typically will depress or deform prior to puncture of the skin. In some cases, the skin may depress enough to prevent the needle from puncturing the skin. In those cases in which the microneedle does puncture the skin, the skin may remain depressed following puncture resulting in a decrease in the effective depth within the skin that the needle reaches. Skin depression is a factor in the effectiveness of a microneedle because the distance that the skin depresses may be a significant percentage of the total length of the microneedle. Further, after a microneedle has punctured the skin, an undesirable amount of the substance delivered through the hollow tip of the microneedle may leak back to the surface of the skin through a weak seal between the needle-skin interface.

In the embodiment shown, delivery device 16 includes a tissue support structure that is configured to decrease the amount of skin depression that occurs prior to skin puncture, to decrease the amount of skin depression that remains after the microneedle is fully extended, and to increase the sealing effect that occurs between the skin and the outer surface of the microneedle. Decreasing skin depression that occurs prior to (or during) puncture allows delivery device 16 to incorporate microneedles of decreased sharpness and to deliver microneedles with less force or velocity than would otherwise be needed. Decreasing skin depression that remains after the microneedle is inserted into the skin allows the microneedles to be delivered deeper into than skin than otherwise would occur with microneedles of a particular length. Further, increasing sealing between the skin and the microneedle shaft may decrease the amount of drug that is leaked to the surface of the skin and is intended to also allow drug to be delivered to the skin through the microneedle at higher pressure and at a higher delivery rate than would possible with less sealing. This enables higher volume intradermal delivery over a shorter period of time than has otherwise been possible. For example, in one embodiment, it is believed that drug delivery device 16 including a tissue support structure as described herein may be able to deliver approximately 0.5 ml of drug in approximately two minutes. In another exemplary embodiment, it is believed that drug delivery device 16 including a tissue support structure as described herein may be able to deliver approximately up to 1 ml of drug in approximately 15-30 seconds.

In the embodiment shown, the tissue support structure includes at least one channel, shown as channels 116 formed through bottom wall 61 and adhesive layer 22, a tensile membrane or rigid wall or backing, shown as the portion of the rigid bottom wall 61 positioned beneath microneedle array 134, and an engagement element, shown as the portion of the adhesive layer 22 adjacent to channels 116. In this embodiment, the portion of bottom wall 61 below forms a structural layer or backing to which adhesive layer 22 is attached. Further, in the embodiment shown in FIGS. 12-16, channels 116 are cylindrical channels (e.g., shaped to have a circular cross section) having a substantially constant diameter along the height of the channel. Further, in the embodiment shown, the diameters of channels 116 are substantially the same as the diameter of the base of the microneedles 142. It should also be clear that in the embodiment shown, adhesive layer operates both as an attachment element providing gross attachment of delivery device 16 to skin 132 and as the engagement element of the tissue support structure.

FIG. 12 shows microneedle array 134 prior to activation with microneedles 142 poised directly above channels 116. As explained above, when delivery device 16 is activated via button 20, torsion rod 106 is released. Prior to activation, U-shaped contact portion 144 of torsion rod 106 is in contact with the upper surface barrier film 86 above microneedle array 134. As shown in FIG. 13, when released, torsion rod 106 applies a downward force to the upper surface barrier film 86 above microneedle array 134. By this arrangement, torsion rod 106 pushes microneedle array 134 downward, moving microneedles 142 through channels 116 and bringing the tips of microneedles 142 into contact with the upper surface of skin 132.

As shown in FIGS. 13 and 14, skin 132 is depressed or deformed a distance D1 by the downward movement of microneedles 142 prior to puncture. It should be noted that the depression distance prior to puncture D1 is exaggerated for illustration purposes. As shown in FIGS. 15 and 16, as microneedles 142 continue to travel downward the upper surface of skin 132 is punctured allowing microneedles 142 to pass into the layers of skin 132 below the surface. Following puncture by microneedles 142, skin 132 rebounds somewhat such that the depression distance of skin 132 following puncture, shown as D2 in FIG. 16, is less than D1. In another embodiment, skin 132 may remain depressed (i.e., does not rebound) following puncture. The amount that skin 132 remains depressed following puncture depends, in part, on the distance between the inner edge of adhesive layer 22 at channel 116 and the shaft 160 of microneedle 142. In addition, with a portion of microneedle 142 positioned within skin 132, there is an interface 158 between skin 132 and the shaft 160 of microneedle 142. As fluid is delivered through central channel 156 of microneedle 142 into skin 132, interface 158 acts as a seal to inhibit or prevent the fluid from leaking back out through the puncture hole to the surface of the skin.

In the embodiment shown, the portion of adhesive layer 22 surrounding and adjacent to channels 116 acts as a support structure by physically limiting the surface deformation and thereby the initial depression of skin 132 depicted by D1 in FIG. 14. The attachment or bond between adhesive layer 22 and skin 132 resists or prevents the inward and downward depression or deformation of skin 132 caused by the downward movement of microneedles 142. In other words, the bond between adhesive layer 22 and skin 132 exerts reaction forces in the skin in response to the penetration of skin 132 by microneedle 142 to resist deformation of the skin. Because adhesive layer 22 is adhered to the outer surface of skin 132 around the periphery of channels 116, adhesive layer 22 tends to maintain the position of the outer surface of skin 132 below channel 116 more precisely than if adhesive layer 22 were not present. In one embodiment, adhesive layer 22 attaches to or anchors the portion of the outer surface of skin 132 adjacent to channel 116 at a fixation point that skin 132 pulls against as the microneedle urges the skin downward away from adhesive layer 22. Adhesive layer 22 geometrically increases the tension or membrane stiffness of the portion of skin 132 below channel 116, and thus, facilitates penetration of skin 132 by microneedle 142. The increased membrane tension results in a decrease in compliance of the portion of the skin below the microneedle, facilitating piercing of the skin by the microneedle.

Further, in the embodiment shown in FIG. 14, because channels 116 surround or encircle microneedle 142 at the point of contact between the tip of microneedle 142 and skin 132, adhesive layer 22 is also adhered to skin 132 adjacent to the entire outer surfaces of microneedles 142. In other words, in the case of channels 116, adhesive layer 22 completely surrounds or encircles each microneedle 142 as microneedle 142 is brought into contact with the skin. The hold of the portion of the outer surface of skin 132 below channel 116 provided by adhesive layer 22 allows microneedle 142 to puncture skin 132 with less depression than if adhesive layer 22 were not present. In one embodiment, the bond between adhesive layer 22 and the skin adjacent to channels 116 may tend to pull skin 132 up towards adhesive layer 22 following puncture thereby decreasing the amount of depression that remains following microneedle insertion. The reinforcement of the tissue provided by adhesive layer 22 also tends to increase the sealing that occurs at interface 158. In addition, as more of the shaft 160 of microneedle 142 becomes embedded in the skin, the length of interface 158 increases, which increases the sealing that occurs along interface 158.

Rigid bottom wall 61 provides a rigid support or anchor for adhesive layer 22 to pull on as adhesive layer 22 acts to resist or prevent the downward depression of skin 132. The effectiveness of adhesive layer 22 as part of a support structure is increased as the strength of the adherence between adhesive layer 22 and the outer surface of skin 132 is increased. The effectiveness of adhesive layer 22 as part of a support structure is also increased as the edge of the adhesive layer at channel 116 is brought closer to shaft 160 of microneedle 142. Thus, in the embodiments of FIGS. 12-16, the cylindrical channel 116 has a diameter minimized to match the diameter of the base of microneedle 142. According to various exemplary embodiments, the diameter of channel 116 is between 1.0 mm and 1.5 mm, preferably is between 1.20 mm and 1.35 mm, and even more preferably is between 1.25 mm and 1.30 mm. In one preferred embodiment, the diameter of channel 116 is 1.27 mm.

As shown in FIG. 15, torsion rod 106 applies a force to microneedle array 134 to hold or maintain the position of microneedle 142 within skin 132 during drug delivery. As shown in FIGS. 12-16, microneedle array 134 includes a body 163, and body 163 of microneedle array 134 includes a lower surface 165. In this arrangement, torsion rod 106 causes lower surface 165 of microneedle array 134 to bear against a portion of the upper surface of bottom wall 61. Thus, bottom wall 61 supports microneedle array 134 while torsion rod 106 holds microneedles 142 in position during drug delivery. Because lower surface 165 of microneedle array 134 does not bear directly on the outer surface of skin 132, skin 132 experiences little or no compression following activation of delivery device 16. In other words, the engagement between the upper surface of bottom wall 61 and lower surface 165 of microneedle array 134 prevents or reduces the amount of compression experienced by skin 132 that may otherwise result if lower surface 165 of microneedle array 134 were to directly contact the outer surface of skin 132. Minimizing compression of skin 132 allows the drug delivered through the tip of microneedle 142 to flow more freely within in the skin beneath microneedle array 134, allowing drug to flow into more layers of the skin than may otherwise result if lower surface 165 of microneedle array 134 were to directly contact the outer surface of skin 132. Allowing the drug to reach more layers of the skin is advantageous for some drug delivery applications. For example, if delivery device 16 is configured for delivery of a vaccine, allowing the vaccine to flow into additional and/or shallower layers of the skin may improve the immune response triggered by the vaccine.

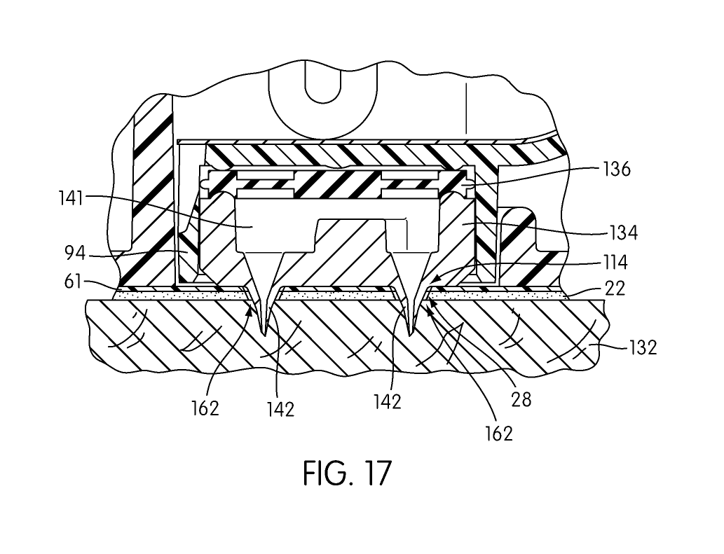

In another embodiment, shown in FIG. 17, holes 114 in bottom wall 61 and holes 28 in adhesive layer 22 have tapered sidewalls such that the holes have a diameter that decreases in the direction toward the outer surface of adhesive layer 22 forming generally cone-shaped channels 162 having tapered sidewalls. In this embodiment, the diameters of channels 162 at the point of contact between adhesive layer 22 and skin 132 are less than in the case of the cylindrical channels. Thus, tapered channel 162 brings the edge of adhesive layer 22 at channel 162 closer to the point of contact between the tip of microneedle 142 and skin 132 than the cylindrical channels 116.

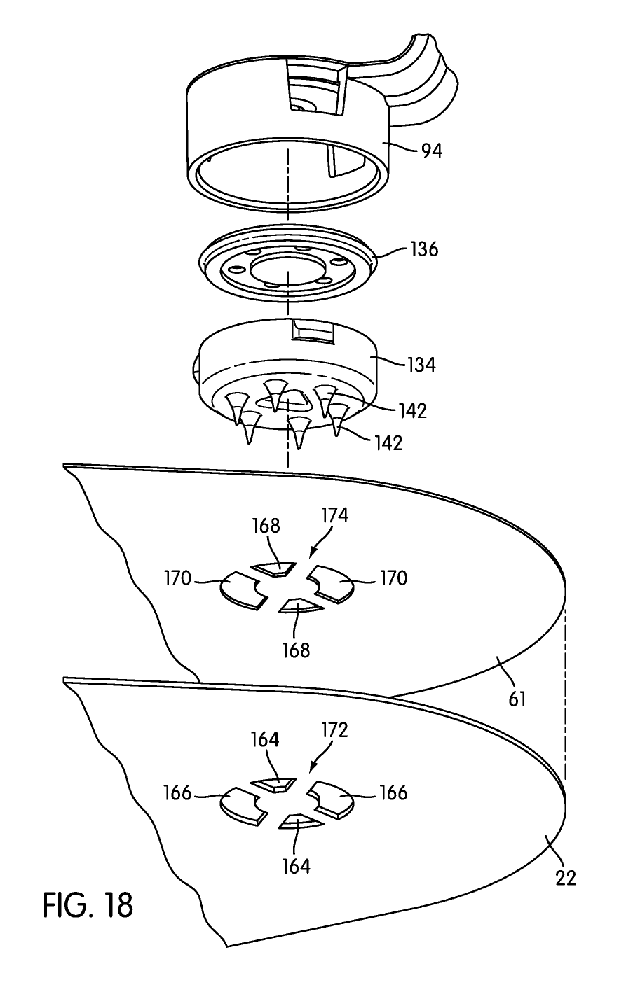

Referring to FIG. 18, another exemplary embodiment of a support structure is shown. In FIG. 18, adhesive layer 22 includes a first pair of holes 164 and a second pair of holes 166. Each hole 164 is sized to receive a single microneedle 142, and each hole 166 is sized to receive two microneedles 142. In this embodiment, rigid bottom wall 61 includes a first pair of holes 168 and a second pair holes 170 that are sized to match holes 164 and 166, respectively. Adhesive layer 22 includes a portion 172 on the interior of holes 164 and 166 that provides for adhesive along at least a portion of the inner edges of microneedles 142. Bottom wall 61 includes a portion 174 that matches the shape of portion 172 and provides support for portion 172 of adhesive layer 22.

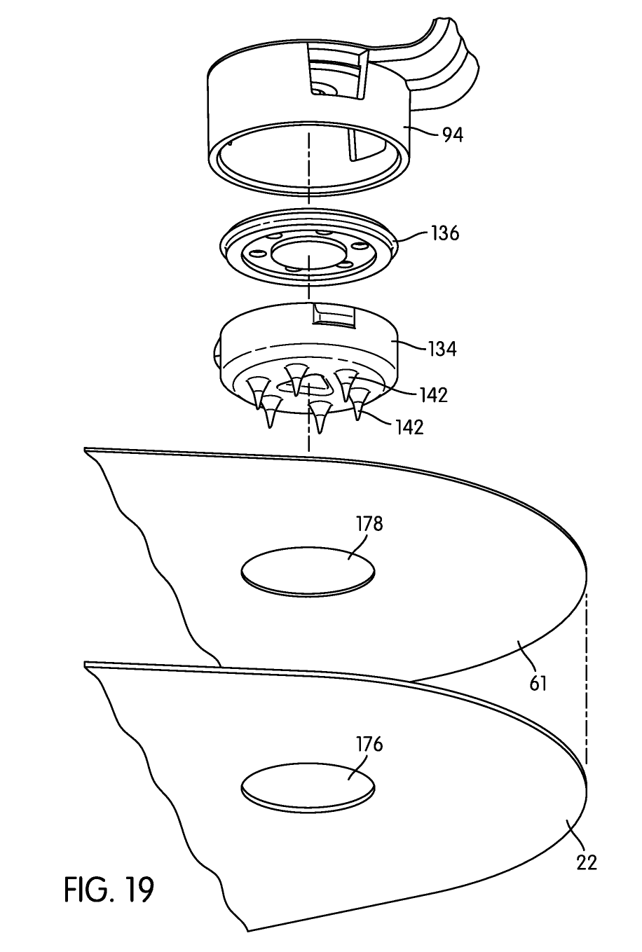

Referring to FIG. 19, another exemplary embodiment of a support structure is shown. In FIG. 19, adhesive layer 22 includes a single hole 176, and bottom wall 61 includes single hole 178 aligned with single hole 176. In this embodiment, hole 176 and hole 178 form a channel that receives all six microneedles 142 of microneedle array 134. In this embodiment, the support provided by adhesive layer 22 is only along the outer edges of microneedles 142. It should be noted that while the tissue support structure embodiments discussed herein include a layer of adhesive to adhere to the skin to provide support to and to resist downward depression of the skin caused by contact with the microneedle, other skin engagement elements may be used that resists downward depression. For example in one embodiment, the lower surface of bottom wall 61 below microneedle array 134 may include hook structures to engage the skin adjacent to channels 116 to resist downward depression or deformation. In another embodiment, the lower surface of bottom wall 61 below microneedle array 134 may include clamp or pinch structures to engage the skin adjacent to channels 116 to resist downward depression or deformation.

Further modifications and alternative embodiments of various aspects of the invention will be apparent to those skilled in the art in view of this description. Accordingly, this description is to be construed as illustrative only. The construction and arrangements of the drug delivery device assembly and the drug delivery device, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

What is claimed is:

1. A drug delivery device for delivering a drug to a subject, the device comprising:
a microneedle configured to facilitate delivery of the drug to the subject, the microneedle including a tip portion, the microneedle moveable from an inactive position to an activated position, wherein when the microneedle is moved to the activated position, the tip portion of the microneedle is configured to penetrate the skin of the subject; and
a tissue support structure comprising:
a channel having a first end and a second end, the channel in axial alignment with the microneedle, wherein at least the tip portion of the microneedle extends past the second end of the channel in the activated position; and
an engagement element positioned adjacent to the channel, the engagement element configured to engage with the skin of the subject such that the engagement element resists deformation of the skin caused by the microneedle as the microneedle moves from the inactive position to the activated position.

2. The device of claim 1, wherein the engagement element comprises an adhesive material, wherein the adhesive material is configured to form a nonpermanent bond to the skin of the subject, the bond being of sufficient strength to increase membrane stiffness in a portion of the skin located beneath the microneedle, the increased membrane stiffness resulting in a decrease in compliance of the portion of the skin facilitating piercing of the skin by the microneedle.

3. The device of claim 2, wherein the tissue support structure further comprises a tensile wall having an upper surface and a lower surface, wherein the adhesive material is coupled to the lower surface of the rigid wall.

4. The device of claim 3, wherein the adhesive material includes a first hole and the tensile membrane includes a second hole aligned with the first hole, wherein the first and second holes define the channel.

5. The device of claim 2, wherein the adhesive material encircles a shaft of the microneedle in the activated position.

6. The device of claim 1, wherein the channel is a cylindrical channel and further wherein the diameter of the channel at the first end is substantially same as a diameter of a base of the microneedle.

7. The device of claim 1, wherein the channel has a circular cross section and further wherein the diameter of the channel at the first end is greater than the diameter of the channel at the second end.

8. The device of claim 6, wherein the channel is tapered between the first and second ends.

9. The device of claim 1, wherein the microneedle is a hollow microneedle having a central channel extending through the tip portion of the microneedle, and further wherein the drug is a liquid drug to be delivered to the subject through the central channel and through the tip portion of the microneedle to the skin of the subject.

10. The device of claim 1, further comprising:
a second microneedle configured to facilitate delivery of the drug to the subject, the second microneedle including a tip portion, the second microneedle moveable from an inactive position to an activated position, wherein, when the second microneedle is moved to the activated position, the tip portion of the second microneedle is configured to penetrate the skin of the subject;
wherein the tissue support structure includes a second channel having a first end and a second end, the second channel in axial alignment with the microneedle, wherein at least the tip portion of the second microneedle extends past the second end of the second channel in the activated position; and
a second engagement element positioned adjacent to the channel, the second engagement element configured to engage with the skin of the subject such that the second engagement element resists deformation of the skin caused by the second microneedle as the second microneedle moves from the inactive position to the activated position.

11. The device of claim 10, wherein both the engagement element and the second engagement element are adhesive materials configured to form nonpermanent bonds to the skin of the subject, the bond being of sufficient strength to resist the deformation of the skin as the first and second microneedles move from the inactive position to the activated position, and further wherein the adhesive materials of the first and second engagement elements encircle shaft portions of the first and second microneedles in the activated position.

12. A drug delivery device for delivering a liquid drug into the skin of a subject, the device comprising:
a drug reservoir for storing a dose of the liquid drug;
a microneedle component including a hollow microneedle, the hollow microneedle including a tip portion and a central channel extending through the tip portion of the hollow microneedle, the microneedle component moveable from an inactive position to an activated position, wherein when the microneedle component is moved to the activated position, the tip portion of the hollow microneedle is configured to penetrate the skin of the subject;
a drug channel extending from the drug reservoir and coupled to the microneedle component such that the drug reservoir is in fluid communication with the tip portion of the hollow microneedle;
an engagement element positioned adjacent to the hollow microneedle in the activated position, the engagement element configured to adhere to the skin of the subject such that the engagement element exerts reaction forces on the skin in a direction opposite to the direction of movement of the microneedle component from the inactive position to the activated position.

13. The drug delivery device of claim 12, wherein the engagement element comprises an adhesive material, and the adhesive material is configured to form a nonpermanent bond to the skin of the subject, the bond being of sufficient strength to resist deformation of the skin as the hollow microneedle moves from the inactive position to the activated position.

14. The drug delivery device of claim 13, further comprising a tensile membrane having an upper surface and a lower surface, wherein the adhesive material is coupled to the lower surface of the tensile wall.

15. The drug delivery device of claim 14, wherein the adhesive material includes a first hole and the tensile membrane includes a second hole aligned with the first hole, wherein the first and second holes define a channel, the channel having a first end and a second end, the channel in axial alignment with the hollow microneedle, wherein at least the tip portion of the hollow microneedle extends past the second end of the channel in the activated position.

16. The drug delivery device of claim 13, wherein the tensile membrane is a rigid wall, wherein the engagement element exerts reaction forces on the skin perpendicular to the movement of the microneedle component, and further wherein the microneedle component is a microneedle array including a plurality of hollow microneedles.

17. The drug delivery device of claim 16, further comprising a plurality of channels each corresponding to one of the plurality of hollow microneedles, each of the plurality channels having a first end and a second end, each of the plurality of channels in axial alignment with one of the plurality of hollow microneedles, wherein at least the tip portion of each hollow microneedle extends past the second end of the respective channel in the activated position.

18. The drug delivery device of claim 17, wherein the engagement element comprises an adhesive material surrounding each of the plurality of channels.

19. A method of delivering a drug to the skin of a subject, the method comprising:
providing a drug delivery device, the drug delivery device comprising:
a dose of the drug to be delivered;
a microneedle;
an attachment element; and
a tissue support structure including a skin engagement element;
attaching the drug delivery device to the skin of the subject via the attachment element;
attaching the skin engagement element to the skin of the subject;
moving the microneedle from an inactive position to an activated position in which a tip portion of the microneedle pierces the skin of the subject;
increasing membrane stiffness in a portion of the skin located beneath the microneedle, the increased membrane stiffness resulting in a decrease in compliance of the portion of the skin facilitating piercing of the skin by the microneedle; and
delivering the dose of the drug to the subject via the microneedle.

20. The method of claim 19, wherein the drug delivery device further comprises an adhesive layer, the adhesive layer being both the attachment element and the skin engagement element.

21. A drug delivery device for delivering a drug to a subject, the device comprising:
a microneedle component having a body and a microneedle, the microneedle configured to facilitate delivery of the drug to the subject, the microneedle including a tip portion, the microneedle moveable from an inactive position to an activated position, wherein when the microneedle is moved to the activated position, the tip portion of the microneedle is configured to penetrate the skin of the subject;
a housing having a bottom wall; and
a channel defined in the bottom wall, the channel having a first end and a second end, the channel aligned with the microneedle;
wherein at least the tip portion of the microneedle extends past the second end of the channel in the activated position;
wherein at least a portion of the body of the microneedle component bears against a surface of the bottom wall in the activated position.

22. The drug delivery device of claim 21, wherein a lower surface of the portion of the body of the microneedle component bears against an upper surface of the bottom wall.

23. The drug delivery device of claim 22, wherein the bottom wall is positioned between the skin of the subject and the lower surface of the portion of the body of the microneedle component.
